(19) United States
(12) Patent Application Publication
(10) Pub. No.: US 2002/0022014 A1
(43) Pub. Date: Feb. 21, 2002

(54) COMPOSITIONS AND METHODS FOR DELOUSING

(76) Inventor: THOMAS BELL, SEATTLE, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 08/986,090

(22) Filed: Dec. 5, 1997

(51) Int. Cl. A61K 31/765; A61K 9/14; A01N 25/32; A01N 25/24; A01N 31/00

(52) U.S. Cl. 424/78.37; 424/406; 424/407; 424/487; 514/724

(57) ABSTRACT

Compositions and methods are disclosed for delousing warm-blooded animals, in need thereof, including humans. The compositions include either a water-soluble polyalkylene glycol or a water-soluble polyalkylene oxide mixed with a polymer or copolymer comprising polyalkylene oxide having carboxylic acid-containing residues. Methods of the invention include topical application to a hair-bearing surface of an effective amount of a composition of this invention, and maintaining the same on the surface for a sufficient period of time to delouse the surface. Further optional steps include washing or rinsing the composition from the surface, as well as combing the hair associated with the treated surface.

### TECHNICAL FIELD

This invention relates generally to compositions and methods of delousing and, more specifically, to topical application of an effective amount of a composition to a lice infested surface for a sufficient period of time to delouse the same.

### BACKGROUND OF THE INVENTION

A louse is a small, flat-bodied, wingless, biting and sucking insect present as an external parasite on various animals, including humans. The human lice genus includes pubic lice (Pediculus Humanus Pubis), body lice (Pediculus Humanus Corporis), and head lice (Pediculus Humanus Capitit). Although related, each have specific characteristics with regard to habit and feeding.

Pubic lice are generally confined to the hairs of the genital region. Body lice may be found on the hair of the axilla, eyebrows, eyelashes, beards, and hairy body surfaces. Both pubic lice and body lice may be acquired from the direct contact with another infested individual, wearing contaminated clothing and from toilet seats, or bed clothes. Both pubic lice and body lice typically occur as the result of crowding or unhygienic conditions.

Head lice are small hard-shelled ectoparasites which cling to hair follicles while feeding, mating and laying eggs. The louse must remain on the head, as it will die within a short period of time when removed. The adult female head louse has a lifespan of about one month and proliferates at an incredible rate. A head louse is ready to mate and reproduce within 10 hours after hatching. Under ideal conditions, a female head louse may produce up to 300 eggs in its lifetime, most commonly on the hair above the ears and the back of the head of an individual, and near the base of hair shafts. The eggs are cemented to the hair with a tenacious protein-like substance. Head lice eggs (commonly referred to as nits) are protected by a chitinous sheath surrounding the eggs which is attached to the hair follicles. These eggs hatch in about seven to nine days and become mature in another week.

As with pubic and body lice, head lice are also transmitted by personal contact, as well as by common use of brushes, combs or headgear. Poor hygienic and grooming habits are known to contribute significantly to the spread of head lice. Thus, head lice infestations are most serious in areas where the inhabitants have both substandard hygienic facilities and practices.

Lice have constituted a “plague” on mankind for many centuries. Infestation of the body by lice is an age-old problem and reference to these pests can be found throughout documented history. For example, lice have been responsible for the spread of typhus, causing decimation of many armies and navies of the military powers from the 15^{th} through the 18^{th} centuries. Even today, lice are still considered as disease vectors and present serious health problems throughout the world. Specifically, lice are vectors for staphylococcal skin infections, such as impetigo and furunculosis, and are also the principle method of transmission for typhus, trench fever and relapsing fever.

In addition to spreading diseases, lice carry a wide variety of bacteria on their exterior surfaces, and their fecal matter transmits disease when it enters the puncture wounds lice inflict during feeding. As the lice feed on human skin, they inject their digestive juices and fecal material into the skin. These materials, as well as the puncture wound itself, cause pruritus, which is painful to the animal host. Therapy which simply kills the lice leaves subcutaneous or intradermal residues which continue to itch for significant time periods after the infestation is extinguished. Furthermore, scratching during and after the episode frequently leads to painful excoriation.

Head lice, pubic lice and body lice are all capable of infesting humans (referred to as pediculosis), the characteristics of which differ markedly within the genus. Head lice cause Pediculosis Captitis in humans, which is characterized by itching and possibly eczematous dermatitis. In long standing and/or neglected cases, scratching may result in marked inflammation, secondary infection by bacteria may occur, resulting in the formation of pustules, crusts and suppuratim, and the hair may become matted and give rise to a disgusting odor. Today, head lice causes Pediculosis Captitis in roughly 2-3% of grade school children in the United States and England.

Body lice cause Pediculosis Corpus in humans. Pediculosis Corpus is a lice infestation characterized by intense itching and, in cases of heavy infestation, by red skin eruptions, mild fever, tiredness, irritability and possibly weakness. Further, pubic lice cause Phthirus Pubis in humans, which is an infestation characterized by itching, especially in the genital or crural regions. Small pale-blue spots resulting from the action of salivary secretion on hemoglobin are characteristic of Phthirus Pubis.

Previous methods for treating pediculosis have not been entirely satisfactory. In the past, chemical agents, including toxic pesticides or insecticides (termed pediculicides) have been used. Unfortunately, these compounds include potent neurotoxins, as well as carcinogens such as DDT, carbaryl, piperonyl butoxide, the synthetic pyrethrin analog permethrin or cholinesterase inhibitors such as neostigmine or prostigmine. These pediculicides range from fairly toxic (pyrethrins) to very toxic (lindane and malathion). All current methods involve the exposure of the scalp and hair to these agents in shampoos or creams, which are then washed off relatively quickly. Because of increased concern about the overall safety of some of the known ectoparasitic toxicants, the search for new and effective pediculicides has intensified.

In addition to safety concerns regarding the current pediculicides, other problems exist with these chemical agents. One drawback is that chemical agents may not achieve the desired efficacy. For instance, lice have developed a significant degree of resistance to lindane. Therefore, natural pyrethrins and synthetic pyrethroids are routinely chosen for use in the treatment of pediculosis. Natural pyrethrins, however, are not without their limitations. Such agents have a poor environmental stability, which is a severe drawback in the treatment of lice. Additionally, natural pyrethrins have low residual action due to this instability. The practical effect is that the use of natural pyrethrins necessitates frequent follow-up treatments. While synthetic pyrethroids are cheaper, more available and more stable than natural pyrethrins, and generally more effective against lice than natural pediculicides, some of these synthetic varients are more toxic to the subject being treated. Again, this poses a safety concern.

While current treatments are generally effective at killing the adult lice and eggs (albeit posing a safety concern), the dead nits remain firmly attached to the hairs after treatment. This is unsettling to the patient, as well a significant cosmetic problem. The only current means of removing dead nits is with a fine toothed comb or forceps. These methods are time consuming and less than certain. Furthermore, if the patient is a child, the child must be nit-free to return to school.

Other forms of treatment are physical in nature and include removal of the hair-infested areas in question, followed by washing with strong soap and optionally one of the pediculicides listed above. Clothing or instruments in contact with the affected body parts should also to be sterilized.

Accordingly, while advances have been made in the field of delousing, further improvements are still needed. For example, compositions and methods are needed which effectively kill the lice, as well as unlock the bond between the lice egg and the hair to ease removal of the eggs. The present invention fulfills these needs and provides further related advantages.

### SUMMARY OF THE INVENTION

In brief, the present invention discloses compositions and methods for delousing hair-bearing warm-blooded animals, including humans, as well as preventing lice infestation. This is accomplished by topically applying to a hair-bearing surface of a warm-blooded animal in need of delousing (or prevention of lice infestation) an effective amount of a composition of this invention, and maintaining the composition on the surface for a sufficient period of time to delouse the surface (or prevent lice infestation).

Suitable time periods for maintaining the composition on the surface are generally greater than twenty seconds, and typically range from twenty seconds to forty minutes for delousing, and from one hour to several hours for preventing infestation. Methods of this invention further include washing or rinsing the treated surface to remove lice eggs, as well as removal of the same by means of a comb.

In one embodiment of this invention, the composition comprises a water-soluble polyalkylene glycol, such as polyethylene glycol, and has a molecular weight ranging from 100,000 to 8,000,000.

In another embodiment, the composition comprises a water-soluble polyethylen eoxide and a polymer or copolymer of polyalkylene oxide having carboxylic acid-containing residues resulting from the polymerization of a monomer selected from acrylic acid and methacrylic acid, and salts thereof. The composition of this invention may also include further optional compounds, such as an antioxidant and/or a pediculicide.

Hair-bearing surfaces which are particularly suitable for treatment by the methods of this invention include, but are not limited to, the scalp and the genitalia, and the hair associated therewith.

These and other aspects of this invention will become evident upon reference to the following detailed description.

### DETAILED DESCRIPTION OF THE INVENTION

As mentioned above, this invention provides methods and compositions for delousing, as well as for preventing lice infestation. As used herein, “delousing” means the killing of lice. Removal of the dead lice from the treated surface may be accomplished by a subsequent removal step, which may take a variety of forms as discussed in greater detail below. An important aspect of this invention is that delousing facilitates the subsequent removal of the dead lice. With respect to the lice eggs or nits, delousing means dissolving the nit cement that holds the eggs to the hair, thus facilitating their subsequent removal.

The methods employ a composition containing either a water-soluble polyalkylene glycol, or a mixture of a water-soluble polyalkylene oxide and a polymer or copolymer of polyalkylene oxide having carboxylic acid-containing residues. In the context of this invention, “water-soluble” means capable of being dissolved in water without the aid of a detergent or similar agent.

A water-soluble polyalkylene glycol is represented by the formula HO—(R—O)ₙ—H. Thus, the polyalkylene glycol contains two terminal hydroxyl groups (OH), with a mid-section composed of repeating alkyleneoxide groups (RO). The letter “n” in the formula represents the number of repeating alkylenoxide units. This number may range from about 5 to several thousand. As the number of repeating units increases, the molecular weight of the polyalkylene glycol will also increase. In a preferred embodiment, the molecular weight of the polyalkylene glycol, in terms of number average molecular weight, is within the range of about 100,000 to about 8,000,000.

In a preferred embodiment, the polyalkylene glycol constitutes from about 0.01 to about 45 weight percent of the composition (based on the total weight of the composition). Thus, the ratio of the weight of polyalkylene glycol to the weight of the entire composition will be between about 0.01 to 45 percent. The composition may be prepared in a concentrated form, with little or no water. In such a composition, polyalkylene glycol will comprise about 45 weight percent of the composition. Alternately, the composition may be prepared, with water present at about 99 percent weight of the composition. In such a composition, polyalkylene glycol will comprise about 0.1 weight percent of the composition. Additionally, the composition may be prepared in a diluted form, with water present in an excess of 99 percent weight of the composition. In such a composition, polyalkylene glycol will comprise about 0.01 weight percent of the composition.

In a preferred embodiment, the alkylene group, R, of the polyalkylene glycol is selected from C₁-C₁₀ alkylene, C₃-C₉ alkylenecycloalkyl, C₇-C₂₀ alkylenearyl, and substituted derivatives thereof. As used herein, the alkylene group includes both straight-chained and branched alkylene groups with the number of carbon atoms ranging from one to ten. Examples of which include, but are not limited to, methylene, ethylene, propylene, isopropylene, tert-butylene and pentylene.

A C₃-C₉ alkylenecycloalkyl includes both straight-chained and branched alkylene groups with the number of carbon atoms ranging from three to nine, less the number of carbon atoms in the cycloalkyl group. The cycloalkyl group is covelantly bonded to a carbon atom of the alkylene group. As used herein, a cycloalkyl group is any cyclic moiety containing carbon and hydrogen, wherein carbon atoms are bonded in a sequential pattern forming a ring system. Examples of which include, but are not limited to, methylenecyclohexane, 2-ethylenecyclopentane and 3-propylenecyclobutane.

A C₇-C₂₀ alkylenearyl includes both straight-chained and branched alkylene groups with the number of carbon atoms ranging from three to nine, less the number of carbon atoms in the aryl group. The aryl group is covelantly bonded to a carbon atom of the alkylene group. As used herein, an aryl group is any cyclic aromatic moiety containing carbon and hydrogen wherein carbon atoms are bonded in a sequential pattern forming an aromatic ring system. Examples of which include, but are not limited to, phenyl-methylene, phenyl-1-ethylene, phenyl-3-tert-butylene, 2-napthyl-1-ethylene and 1-napthyl-2-propylene.

In the context of this invention, a water-soluble polyalkylene oxide is represented by the formula R₁O—(R₂—O)ₙ—H, wherein R₁ and R₂ are independently selected from a straight-chained C₁-C₁₀ or branched alkyl groups. The polyalkylene oxide contains a terminal alkoxy group (R₁O), with a mid-section composed of repeating alkyleneoxide groups (R₂—O) and a terminal hydroxy group (OH). The letter “n” in the above formula represents the number of repeating alkylenoxide units and may range from about five to several thousand. As used herein, alkoxy means any straight-chained or branched C₁-C₁₀ alkyl bonded directly to an oxygen atom. Examples of which include, but are not limited to, methoxy, ethoxy and pentoxy moieties.

A polymer or copolymer of polyalkylene oxide having carboxylic acid-containing residues is represented by the formula R₅O—(R₆(COOH)O)ₙ—H. The letter “n” in the formula represents the number of repeating alkylene carboxylic acid units, and may range from about five to several thousand. Thus, the polymer or copolymer contains a terminal alkoxy group (R₅O), with a mid-section composed of repeating alkylene oxide groups with carboxylic acid-containing residues (R₆(COOH)O) and a terminal hydroxy group (OH). R₅ of the alkoxy group is a straight-chained or branched C₁—C₁₀ alkyl group. The repeating monomer, (R₆(COOH)O), can be either acrylic acid or methacrylic acid wherein R₆(COOH) is CH₂=CH(COOH) when the monomer is acrylic acid, and wherein R₆(COOH) is CH₂=C(CH₃)(COOH) when the monomer is methacrylic acid.

Thus, in another embodiment of this invention, the composition contains polyalkylene oxide and a polymer or copolymer of polyalkylene oxide having carboxylic acid-containing residues. In a preferred embodiment, the combination of polyalkylene oxide and the polymer or copolymer together constitutes from about 0.1 to about 99 weight percent of the composition, based on the total weight of the composition. Thus, the ratio of the combined weight of polyalkylene oxide and the polymer or copolymer to the weight of the entire composition will be between about 0.1 to 99 percent. The composition may be prepared in a concentrated form, with little or no water. In such a composition, polyalkylene oxide and the polymer or copolymer together will comprise up to about 99 weight percent of the composition. Alternately, the composition may be prepared with water present in an amount up to about 99 percent weight of the composition. In such a composition, polyalkylene oxide and the polymer or copolymer together will comprise up to about 0.15 weight percent of the composition. Additionally, the composition may be prepared in a diluted form, with water present in excess of 99 percent weight of the composition. In such a composition, polyalkylene oxide and the polymer or copolymer together will comprise about 0.1 weight percent of the composition.

In one embodiment, the compositions of this invention further comprises an antioxidant. As used herein, antioxidant refers to an agent that inhibits or slows down oxidation and thus prevents rancidity of any oils which may be present in the composition or prevents the deterioration of other materials which may be present in the composition through oxidative processes. The specific antioxidant employed will depend on the desired composition. In a further preferred embodiment, the antioxidant is methylparaben. As used herein, methylparaben is 4-Hydroxybenzoic acid methyl ester.

Alternatively, the compositions may further include an optional pediculicide. Such agents are well known in the art and are present in many commercial delousing compositions, including (without limitation) nonoxynol, pyrethrins, piperonyl butoxide, permethrin, malathion, carbaryl, cuprex, phenothrin, DDT, neostigmine, prostigmine and lindane.

As used herein, nonoxynol refers to any of the series of octoxynols. Octoxynol may be represented by the formula (CH₃)CCH₂C(CH₃)₂(C₆H₆)—O—(CH₂CH₂O)ₙH where the letter “n” in the formula represents the number of repeating ethylene oxide units which may range from about two to about forty.

Pyrethrins refer to the active insecticidal constituents of pyrethrum flowers. In one embodiment, the pyrethrin is 2,2-dimethyl-3-(2-methyl-1-propenyl)cyclopropanecarboxylic acid 2-methyl-4-oxo-3-(2,4-pentadienyl)-2-cyclopenten-1-yl ester. In another embodiment, the pyrethrin is 3-(3-methoxy-2-methyl-3-oxo-1-propenyl)-2,2-dimethylcyclopropanecarboxylic acid 2-methyl-4-oxo-3-(2,4-pentadienyl)-2-cyclopenten-1-yl ester.

Cuprex is a combination of tetrahydronapyhalene and copper oleate; carbaryl is 1-naphthalenol methylcarbamate; DDT is 1,1′-(2,2,2-trichloroethylidene)bis[4-chlorobenzene]; neostigmine is 3[[(dimethylamino)carbonyl]oxy]-N,N,N-trimethylbenzenaminium; piperonyl butoxide is 5-[[2-(2-butoxyethoxy)ethoxy]methyl]-6-propyl-1,3-benzodioxole; malathion is [(dimethoxyphosphinothioyl)thio]butanedioic acid diethyl ester; lindane is 1α, 2α, 3β, 4α, 5α,6β-hexachlorocyclohexane; permethrin is 3-(2,2-dichloroethenyl)-2,2-dimethylcyclopropanecarboxylic acid (3-phenoxyphenyl)methyl ester; and phenothrin is 2,2-dimethyl-3-(2-methyl-1-propenyl)cyclopropanecarboxylic acid (3-phenoxyphenyl)methyl ester.

The compositions of this invention may be prepared by combining the various ingredients, with stirring and optionally with mild heating in order to form a homogenous mixture. As used herein, mild heating means heating the ingredients to a temperature less than 80° C. Homogeneous mixture means a mixture which is uniform in structure and composition. In a preferred embodiment, the ingredients are heated to about 25° C.

Alternatively, suitable compositions are commercially available, although not recognized for use in the practice of the present invention. For example, Wallace-O'Farrell Inc. sells a preferred composition under the trademark Slippery Stuff® gel. Slippery Stuff® gel is sold as a glycerine-free, hygienic, sterile, water-based, water-soluble, pharmaceutical lubricant.

The methods of the invention provide for contacting a composition as described above, with a surface. The surface may be infested with lice, infested with nits, infested with lice and nits or free of lice and nits. If the composition is applied to a surface free of lice and nits, the composition is applied to prevent the infestation of lice, the infestation of nits, or the infestation of lice and nits. If the composition is applied to a surface that is infested with lice, the composition is applied in order to kill and remove the lice. If the composition is applied to a surface that is infested with nits, the composition is applied in order ease subsequent removal of the nits from the surface, whether dead or alive.

In one embodiment, the surface to which the composition is to be applied is a human body. Lice are found, on humans, most commonly, where hair is found on the body. Such areas include the scalp and hair on the scalp, the hairs of the genital region, the hair of the axilla, eyebrows, eyelashes, beards, and the body surface of hairy individuals. In one preferred embodiment, the surface is the head and hair on the head. In another embodiment, the surface is hair-bearing surface on the body of a human. In a further embodiment, the surface is the genital region of a human.

The delousing methods of the invention are achieved by topically applying a composition as described above. The composition may be topically applied to the surface by, for example, pouring the composition on the surface, spraying the composition on the surface, and/or rubbing the composition over the surface. When the composition is topically applied as described above, the composition should thoroughly saturate the surface. In order to thoroughly saturate the surface with the composition, a sufficient or adequate amount of composition should be employed.

The composition should remain in contact with the surface for a period of time to effectively delouse the same. To kill lice, the contact will generally be at least about twenty-five seconds. To remove lice eggs, the contact time will be at least about twenty-five minutes. To prevent lice infestation, the composition should remain on the surface for several hours, and the composition should be reapplied to the surface every several hours.

In a further embodiment, the method of this invention comprises the further step of removing the dead lice and/or nits from the treated surface. This may be accomplished by combing the hair after the composition has been topically applied. In another embodiment, the surface is washed and/or rinsed after the composition has been topically applied. As used herein, washing means applying soap, a detergent, a bleaching agent or a surfactant to the surface, preferably in the presence of water or another liquid, by immersing, dipping, rubbing or scrubbing, thereby cleansing the surface. As used herein, rinse means to pass water or another liquid over the surface thereby cleansing the surface.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for delousing comprising topically applying to a hair-bearing surface of a warm-blooded animal in need of delousing an effective amount of a composition comprising a water-soluble polyalkylene glycol, and maintaining the composition on the surface for a sufficient period of time to delouse the surface.

2. The method according to claim 1 wherein the polyalkylene glycol comprises a plurality of alkylene groups, wherein the alkylene groups are selected from C₁-C₁₀ alkylene, C₃-C₉ alkylenecycloalkyl, C₇-C₂₀ alkylenearyl, and substituted derivatives thereof, and the polyalkylene glycol has a molecular weight between 100,000 and 8,000,000.

3. The method according to claim 1 wherein the polyalkylene glycol is polyethylene glycol.

4. The method according to claim 3 wherein the polyethylene glycol has a molecular weight between 100,000 and 8,000,000.

5. The method according to claim 1 wherein the warm-blooded animal is a human.

6. The method according to claim 1 wherein the surface is the scalp of the animal.

7. The method according to claim 1 wherein the surface is the genitalia of an animal.

8. The method according to claim 1 wherein the polyalkylene glycol comprises from 0.01 to 45 weight percent of the composition.

9. The method according to claim 1 wherein the composition further comprises an antioxidant.

10. The method according to claim 9 wherein the antioxidant is methyl paraben.

11. The method according to claim 1 wherein the composition further comprises an agent selected from the group consisting of nonoxynol, pyrethrins, piperonyl butoxide, permethrin, malathion, carbaryl, cuprex, phenothrin, DDT, neostigmine, prostigmine, thepyrethrins, and lindane.

12. The method according to claim 1 wherein the composition is in the form of a gel.

13. The method according to claim 1 wherein the composition is maintained on the surface for a period of time greater than twenty seconds.

14. The method according to claim 1 wherein the composition is maintained on the surface for a period of time greater than twenty minutes.

15. The method according to claim 1 wherein the composition is maintained on the surface for a period of time greater than ten hours.

16. The method according to claim 1 further comprising washing or rinsing the surface.

17. The method according to claim 1 further comprising combing the surface.

18. A method for delousing comprising topically applying to a hair-bearing surface of a warm-blooded animal in need of delousing an effective amount of a composition comprising a water-soluble polyalkylene oxide and a polymer or copolymer of polyalkylene oxide having carboxylic acid-containing residues resulting from the polymerization of a monomer selected from acrylic acid, methacrylic acid and salts thereof, and maintaining the composition on the surface for a sufficient period of time to delouse the surface.

19. The method according to claim 18 wherein the polymer is selected from polyacrylic acid and salts thereof.

20. The method according to claim 18 wherein the copolymer further contains residues resulting from the polymerization of a monomer selected from acrylate ester and methacrylate ester.

21. The method according to claim 20 wherein the number of acrylic acid and methacrylic acid residues is greater than the number of acrylate ester and methacrylate ester residues.

22. The method according to claim 18 wherein the polyalkylene oxide having carboxylic acid-containing residues comprises a plurality of alkylene groups wherein the alkylene groups are independently selected from C₁-C₁₀ alkylene, C₃-C₉ alkylenecycloalkyl, C₇-C₂₀ alkylenearyl, and substituted derivatives thereof.

23. The method according to claim 18 wherein the polyalkylene oxide and the polymer or copolymer together constitute from about 0.1 to 99 weight percent of the entire composition.

24. The method according to claim 18 wherein the polymer or copolymer constitutes from about 0.1 to about 85 weight percent of the composition.

25. A method for delousing comprising topically applying to a hair-bearing surface of a warm-blooded animal in need of delousing an effective amount of a composition, and maintaining the composition on the surface for a sufficient period of time to delouse the surface, wherein the composition is a gel product sold under the trademark Slippery Stuff®.

26. The method according to claim 25 wherein the warm-blooded animal is a human.

27. The method according to claim 25 wherein the surface is the scalp of the animal.

28. The method according to claim 25 wherein the surface is the genitalia of the animal.

29. The method of claim 25 further comprising washing or rinsing the surface.
